(19) United States
(12) Patent Application Publication
(10) Pub. No.: US 2007/0190003 A1
(43) Pub. Date: Aug. 16, 2007

(54) Cosmetic composition comprising a particular ester, and uses thereof

(76) Inventor: Pascal Arnaud, I'Hay les Roses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/565,635

(22) PCT Filed: Jul. 25, 2003
(86) PCT No.: PCT/FR03/02357

§ 371 Date: Jul. 17, 2006

(51) Int. Cl. A61K 8/37

(52) U.S. Cl. 424/64

(57) ABSTRACT

The invention relates to a composition comprising a physiologically acceptable medium containing a fatty phase comprising at least one ester resulting from the reaction of a polyol with a carboxylic acid of formula (I) below: 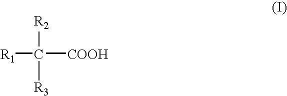
in which R₁, R₂ and R₃ are radicals independently chosen from optionally functionalized alkyl, aryl and aralkyl radicals, and combinations thereof. The invention also relates to the use of an ester of formula (I) above in a cosmetic composition with staying power properties, especially of the colour, and/or gloss and/or comfort and/or migration resistance properties.

The present invention relates to a composition, especially a makeup or care composition for the skin, including the scalp, of either the human face or body, and for human lips or integuments, for instance the hair, the eyelashes, the eyebrows or the nails, comprising a physiologically acceptable medium that contains a fatty phase comprising a particular ester. This composition has noteworthy cosmetic properties, in particular in terms of staying power, and gives the makeup or the care treatment gloss and/or comfort and/or migration resistance properties.

The composition of the invention may in particular be in the form of a makeup product for keratin materials (skin, lips or integuments) in particular having non-therapeutic care and/or treatment properties. It is especially a lipstick, a lip gloss, a foundation, a loose or compact powder, a makeup rouge, an eyeshadow, a makeup base, a concealer product, a tattoo product, a mascara, an eyeliner, a nail varnish, an artificial skin-tanning product, a hair colouring product or a haircare product.

The use of silicone compounds in cosmetic compositions, especially makeup compositions, is known to formulators. Mention may be made, for example, of patent EP-A-0 407 205, which describes a composition comprising a combination of a silicone gum and a silicone oil, which gives the composition good staying power, spreading and comfort properties.

Specifically, these compounds give cosmetic compositions excellent sensory properties, especially a non-greasy feel, and spreading and slip properties, and especially enable a particularly uniform film to be obtained on the skin.

It is also known practice to use these silicone compounds in order to increase the staying power of cosmetic compositions, in particular of makeup, and/or to reduce their migration.

The term “migration” means the running of the composition (and in particular of the colour) beyond the initial applied line of the makeup, resulting in an unattractive effect. Migration is often mentioned by women as being a major defect of standard lipsticks and eyeshadows.

The problems of poor staying power are characterized by a modification of the colour (changing or fading of the colour) generally following an interaction with the sebum and/or sweat secreted by the skin in the case of foundations and face powders, or an interaction with saliva in the case of lipsticks. This obliges the user to reapply makeup very regularly, which may constitute a loss of time.

However, silicone compounds may have problems of compatibility (solubility) with other hydrocarbon-based compounds conventionally used in cosmetic compositions, which limits their use.

Moreover, cosmeticians are currently seeking starting materials that show optimum tolerance with respect to keratin materials, while at the same time being environmentally friendly, which is not always the case for silicone compounds.

The Applicant has found, surprisingly, that the particular esters according to the invention have properties equivalent to those of silicone compounds while at the same time having broader compatibility (solubility). This allows greater diversity in formulations. Furthermore, the esters according to the invention show greater harmlessness with respect to keratin materials and may thus be formulated as a supplement to or a replacement for silicone compounds.

The use of such esters allows the production of compositions with good cosmetic and sensory properties, especially in terms of staying power, in particular of the colour, migration resistance, comfort and absence of irritation to keratin materials, and that have a more or less glossy appearance adapted to the desire of consumers.

Document EP 1 040 814 teaches nail varnish compositions containing an acrylic polymer, the ester of tert-butylic acid and of 2,2,4-trimethyl-1,3-pentanediol, and a second organic solvent. These varnish compositions have good nail adhesion and glossy properties and are easy to remove.

Cosmetic compositions containing monoesters or diesters of neoacid and of diols have been described in patents U.S. Pat. Nos. 4,243,657 and 4,224,311. These compositions allow an improvement in the mechanical strength of the hair on brushing and drying. They also show better stability to oxidation.

Finally, document WO 03/026,698 describes diol diesters. These diesters are esters of neopentanoic acid with non-branched alkylene oxides of formula HO(CₙH₂ₙO)ₘH such that n is equal to 2 or 3 and m is between 2 and 4.

A first subject of the present invention is a composition comprising a physiologically acceptable medium containing a fatty phase comprising at least one ester resulting from the reaction of a polyol with a carboxylic acid of formula (I) below: 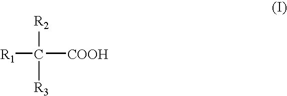
in which R₁, R₂ and R₃ are radicals independently chosen from optionally functionalized alkyl, aryl and aralkyl radicals, and combinations thereof, characterized in that the said polyol is chosen from polyols comprising one carbon atom, located alpha to the carbon bearing an alcohol function, which is trisubstituted with radicals chosen, independently of each other, from alkyl, aryl and aralkyl radicals and combinations thereof, at least one of the alkyl, aryl and aralkyl radicals containing at least one alcohol function, the said polyol not being 2,2,4-trimethyl-1,3-pentanediol.

A second subject of the present invention is a composition comprising a physiologically acceptable medium containing a fatty phase comprising at least one ester resulting from the reaction of a polyol with a carboxylic acid of formula (I) below: 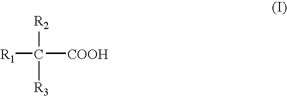
in which R₁, R₂ and R₃ are radicals independently chosen from optionally functionalized alkyl aryl and aralkyl radicals, and combinations thereof, the said acid containing from 5 to 9 carbon atoms, and the said polyol not being a compound of formula HO(CₙH₂ₙO)ₘH such that n is equal to 2 or 3 and m is between 2 and 4.

According to these first two subjects, the term “ester”, according to the invention, means, depending on the case, a monoester, a diester, a triester or, more generally, a polyester.

A third subject of the present invention is a composition comprising a physiologically acceptable medium containing a fatty phase comprising at least one ester resulting from the reaction of a polyol with a carboxylic acid of formula (I) below: 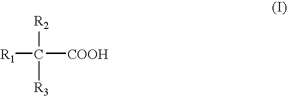
in which R₁, R₂ and R₃ are radicals independently chosen from optionally functionalized alkyl, aryl and aralkyl radicals, and combinations thereof, the said ester comprising at least three ester functions.

According to the three subjects of the invention, the term “at least one ester” means one or more esters.

The term “physiologically acceptable” means non-toxic and capable of being applied to human skin, integuments or lips.

The physiologically acceptable medium of the composition according to the invention is in particular cosmetically acceptable, i.e. it has a pleasant taste, feel, appearance and/or odour, and may be applied for several days over several months.

The term “functionalized” means a radical comprising a hetero atom, i.e. an atom other than a carbon atom (such as an oxygen, nitrogen, sulfur or fluorine atom) or a functional group such as a carboxyl —COOH, hydroxyl —OH, amide —NHR or ester —COOR group, R representing an alkyl radical.

A subject of the present invention is also the use of at least one of the esters described above in a cosmetic composition, as an agent for giving the said composition staying power properties, especially of the colour, and/or gloss and/or comfort and/or migration-resistance properties.

Another subject of the present invention is the use of at least one of the esters described above in a cosmetic composition with staying power properties, especially of the colour, and/or gloss and/or comfort and/or migration-resistance properties.

The present invention also relates to a cosmetic process for giving a film of cosmetic composition staying power properties, especially of the colour, and/or gloss and/or comfort and/or migration-resistance properties, which consists in introducing into the said composition at least one of the esters described above.

The ester of the composition according to the invention may result from a total or partial esterification.

A total ester is an ester not comprising any free OH groups, since all of the alcohol functions of the said polyol have reacted with an acid molecule of formula (I), and since the acid of formula (I) does not comprise a hydroxyl group in its structure.

A partial ester comprises at least one OH group, which may be an alcohol function of the polyol that has not reacted with an acid function of the acid of formula (I). A partial ester may also result from the total reaction of a polyol with an acid of formula (I) such that at least one of the radicals R₁, R₂ and R₃ contains a hydroxyl group.

The ester of the composition according to the invention results from the reaction of a carboxylic acid of formula (I) with a polyol.

Carboxylic Acid

According to the three subjects of the invention, the carbon located alpha to the acid function of the carboxylic acid of formula (I) is trisubstituted with a hydrocarbon-based group. This carbon atom is thus directly bonded to 4 carbon atoms and is termed a “neo” carbon. An acid comprising such a structure is termed a “neo” acid and will be referred to as such in the text hereinbelow. The radicals R_{1, R}₂ and R₃ are advantageously independently chosen from saturated alkyl radicals.

The “neo” carboxylic acid may be a monoacid or a polyacid, i.e. at least one of the radicals R₁, R₂ or R₃ contains a —COOH functional group. The carboxylic acid is preferably a monoacid.

The polyol may be a diol, a triol or a tetraol.

According to the second subject of the invention, the carbon located alpha to the acid function of the carboxylic acid of formula (I) is trisubstituted with radicals independently chosen from saturated or unsaturated, branched or non-branched aliphatic hydrocarbon-based radicals especially containing from 1 to 5 carbon atoms, on condition that the number of carbon atoms in the carboxylic acid of formula (I) is from 5 to 9.

The number of carbon atoms in the carboxylic acid of formula (I) is preferably from 5 to 7.

The radicals R₁, R₂ and R₃ are advantageously independently chosen from saturated alkyl radicals containing from 1 to 5 carbon atoms. The radicals R₁, R₂ and R₃ are especially independently chosen from methyl, ethyl, propyl, isobutyl, isopropyl, butyl and pentyl, on condition that the number of carbon atoms in the carboxylic acid of formula (I) is from 5 to 9.

According to the second subject of the invention, a “neo” carboxylic acid chosen from neopentanoic acid of formula CH₃—C(CH₃)₂—COOH, neohexanoic acid and neoheptanoic acid of formula C₃H₇—C(CH₃)₂—COOH is preferentially used. Neopentanoic acid is preferred, such that the radicals R₁, R₂ and R₃ are methyl radicals.

According to the first and third subjects of the invention, the carbon located alpha to the acid function of the carboxylic acid of formula (I) is trisubstituted with radicals independently chosen from alkyl, aryl and aralkyl radicals, and combinations thereof, these radicals possibly being identical or different.

The term “alkyl radical” means a saturated or unsaturated, branched or non-branched aliphatic hydrocarbon-based chain especially containing from 1 to 28 carbon atoms.

The term “branched” means at least one pendant hydrocarbon-based chain containing at least one carbon atom.

The term “aryl radical” means a radical derived from an aromatic cyclic compound by elimination of a hydrogen atom, for instance phenyl or tolyl radicals.

The term “aralkyl radical” means an alkyl chain substituted with an aryl radical, for example of the type R′—C₆H₅, R′ being a C₁-C₅ alkyl, for instance benzyl or phenethyl radicals.

These alkyl, aryl or aralkyl radicals may be functionalized, which means that they may comprise in their structure a hetero atom, i.e. an atom other than a carbon atom (such as an oxygen, nitrogen, sulfur or fluorine atom) or a functional group such as a carboxyl —COOH, hydroxyl—OH, amide—NHR or ester—COOR group, R representing an alkyl.

Preferentially, the radicals R₁, R₂ and R₃ are independently chosen from unfunctionalized alkyl, aryl or aralkyl radicals.

According to the first and third subjects of the invention, the radicals R₁, R₂ and R₃ are advantageously independently chosen from saturated alkyl radicals, preferably from saturated C₁-C₁₅ and better still C₁-C₆ alkyl radicals, for instance methyl, ethyl, propyl, isopropyl, butyl, isobutyl, pentyl, isopentyl or hexyl radicals.

According to the first and third subjects of the invention, the “neo” carboxylic acid advantageously contains a total number of carbon atoms ranging from 5 to 30, better still from 5 to 15 and even better still from 5 to 10.

According to the first and third subjects of the invention, a “neo” carboxylic acid chosen from neopentanoic acid of formula CH₃—C(CH₃)₂—COOH, neohexanoic acid, neoheptanoic acid of formula C₃H₇—C(CH₃)₂—COOH and neodecanoic acid of formula C₆H₁₃—C(CH₃)₂—COOH, and mixtures thereof, is preferentially used.

More preferentially, the “neo” carboxylic acid is neopentanoic acid.

Polyol

According to a first subject of the invention, the polyol is chosen from “neo” polyols, i.e. polyols containing a carbon atom, located alpha to the carbon bearing an alcohol function, which is trisubstituted with radicals independently chosen from alkyl, aryl and aralkyl radicals and combinations thereof, at least one of the alkyl, aryl and aralkyl radicals containing at least one alcohol function. This carbon atom directly linked to four other carbon atoms is thus a “neo” carbon as defined above.

Advantageously, the “neo” polyol contains a carbon atom located alpha to the carbon bearing one of the alcohol functions that is trisubstituted with radicals independently chosen from saturated alkyl radicals, preferably saturated C₁-C₁₅ and better still C₁-C₆ alkyl radicals, at least one of the alkyl radicals containing at least one alcohol function.

Among the “neo” polyols chosen in particular are trimethylolpropane of formula HOCH₂—C(C₂H₅) (CH₂OH)₂, pentaerythritol of formula HOCH₂—C(CH₂OH)₂—CH₂OH and neopentyl glycol, and mixtures thereof.

The “neo” polyol advantageously contains a number of carbon atoms ranging from 5 to 20 and preferably from 5 to 10. The polyol may be a diol, a triol or a tetraol.

The neodiol chosen in particular is neopentyl glycol of formula HOCH₂—C(CH₃)₂—CH₂OH.

According to a third subject, the ester comprises at least three ester functions and is obtained by reacting a polyol with a carboxylic acid of formula (I).

The polyol may comprise two or more hydroxyl groups, and the acid of formula (I) may comprise radicals R_{, and/or R}₂ and/or R₃ functionalized with ester groups, or alternatively the radicals R₁, R₂ and/or R₃ are functionalized with an ester group.

When only one of the hydroxyl groups of the polyol reacts with the acid of formula (I), the radicals R₁, R₂ and/or R₃ are functionalized with two ester groups.

If the polyol comprises two hydroxyl groups or if only two of the hydroxyl groups of the polyol each react with an acid of formula (I), then at least one of the acids of formula (I) that has reacted with the polyol comprises an ester group.

Finally, if the polyol comprises at least three hydroxyl groups, the acid of formula (I) that has reacted with each of the hydroxyl groups of the polyol does not necessarily comprise at least one radical R₁, R₂ and/or R₃ functionalized with an ester group.

According to the second or the third subject of the invention, the polyol may thus comprise a number of carbon atoms ranging from 2 to 20 and preferably from 3 to 10. The polyol may be a diol, a triol or a tetraol. The polyol is preferably an aliphatic polyol whose skeleton is saturated.

According to the second subject of the invention, the polyol may be chosen from ethylene glycol, propylene glycol, butylene glycol, polypropylene glycol, polyethylene glycols other than a compound of formula HO(CₙH₂ₙO)ₘH such that n is equal to 2 or 3 and m is between 2 and 4, glycerol, diglycerol, triglycerol, isopentyldiol and sorbitol, and mixtures thereof.

According to the third subject of the invention, the polyol may be chosen from ethylene glycol, propylene glycol, butylene glycol, polypropylene glycols, polyethylene glycols, glycerol, diglycerol, triglycerol, isopentyldiol and sorbitol, and mixtures thereof.

According to the second or third subject of the invention, the polyol may be chosen from the “neo” polyols as described above, for example trimethylol-propane, pentaerythritol or neopentyl glycol, or mixtures thereof.

Ester

Preferably, the ester of the composition according to the invention is in liquid form at room temperature (25° C.) and atmospheric pressure (760 mm Hg r 1.01×10⁵ Pa).

This ester is in particular an oil. The term “oil” means any liquid non-aqueous medium that is insoluble in water at room temperature and atmospheric pressure.

According to the first subject of the invention, the ester used in the composition of the invention may be advantageously chosen from neopentyl glycol dineopentanoate (C₁₅) and neopentyl glycol dineoheptanoate (C₁₉), and mixtures thereof. (The Cₙ in parentheses represents the total number of carbon atoms in the mentioned compound.) According to the second subject of the invention, the ester used in the composition of the invention may be advantageously chosen from glyceryl trineopentanoate (C₁₈) and glyceryl trineoheptanoate (C₂₄), and mixtures thereof. (The Cₙ in parentheses represents the total number of carbon atoms in the mentioned compound).

According to the third subject of the invention, the ester is a polyester and comprises at least three ester functions —COO—. It may be a triester, a tetraester or a pentaester. The polyester comprising at least three ester functions results from the reaction of a carboxylic acid of formula (I) with a polyol.

The ester of the composition may result from a total or partial esterification (in the latter case, the ester comprises one or more free —OH functions). The ester advantageously contains no free hydroxyl groups, in the sense that all of the hydroxyl groups of the polyol have reacted with a carboxylic acid molecule not containing any hydroxyl groups.

According to the third subject of the invention, the carboxylic acid of formula (I) may be a monocarboxylic acid or a polycarboxylic acid and the polyol may be a diol, a triol or a tetraol, the said acid and the said polyol being chosen such that their reaction leads to a total or partial ester comprising at least three ester functions.

The polyester preferably results from the reaction of a monocarboxylic acid of formula (I) with a triol (polyol comprising at least three hydroxyl groups). The polyester is preferably a total ester in the sense that it does not comprise any free —OH functions.

The polyester is, for example, glyceryl trineodecanoate (C₃₃).

The ester of the composition according to the present invention may represent from 0.1% to 99.9%, preferably from 1% to 99% and better still from 5% to 90% of the total weight of the composition. It represents, for example, from 5% to 60% of the total weight of the composition.

Advantageously, the ester is in an amount sufficient to give the composition gloss and/or staying power and/or migration-resistance and/or comfort properties.

This ester may be prepared according to the methods known to those skilled in the art. Mention may be made, for example, of the following documents in which the synthesis of this compound is described: Pawlenko Justus, Liebigs Ann. Chem. 663 (1963); M. Fefer and A. J. Rutkowski, Jaocs 45, 5-10 (1968); B. Paulsen, Chem. Ber. 104, 1281-1294 (1971); V. A. Bochkova, J. Appl. Chem. USSR 46, 1929-1932 (1973) and patents U.S. Pat. Nos. 3,441,600 and 3,523,084 by the company Sinclair Research Inc.

The amounts of the various ingredients in the composition according to the invention are given as weight percentages relative to the total weight of the said composition.

The composition according to the invention may also comprise at least one colouring agent, which may be chosen from water-soluble or liposoluble dyes, pigments, nacres and nacreous pigments, and mixtures thereof.

The term “pigments” should be understood as meaning white or coloured particles that are insoluble in the liquid fatty phase of the composition, which are intended to colour and/or opacify the composition.

The terms “nacres” and “nacreous pigments” should be understood as meaning iridescent particles, produced especially by certain molluscs in their shell or alternatively synthesized, which are insoluble in the liquid fatty phase of the composition.

The term “dyes” should be understood as meaning generally organic compounds that are soluble in fatty substances such as oils or in an aqueous-alcoholic phase.

The liposoluble dyes are, for example, Sudan red, D&C Red No. 17, D&C Green No. 6,β-carotene, soybean oil, Sudan brown, D&C Yellow No. 11, D&C Violet No. 2, D&C Orange No. 5, quinoline yellow, annatto and bromo acids.

The water-soluble dyes are, for example, beetroot juice, methylene blue and caramel. The liposoluble and water-soluble dyes may represent from 0.001% to 20% and better still from 0.1% to 6% of the total weight of the composition.

The pigments may be white or coloured, mineral and/or organic, and coated or uncoated. Among the mineral pigments that may be mentioned are titanium dioxide, optionally surface-treated, zirconium oxide and cerium oxide, and also zinc oxide, iron oxide (black, yellow, brown or red) or chromium oxide, manganese violet, ultramarine blue, chromium hydrate and ferric blue. Among the organic pigments that may be mentioned are carbon black, pigments of organic barium, strontium, calcium or aluminium lake type, including those submitted for certification by the Food and Drug Administration (FDA) (for example D&C or FD&C) and those exempt from FDA certification, for instance lakes based on cochineal carmine. The pigments may be present in the composition in a proportion of from 0.05% to 40% and preferably in a proportion of from 2% to 20% of the weight of the final composition, for a non-pulverulent composition. For a pulverulent composition, they may represent up to 70% of the total weight of the composition.

The nacres or nacreous pigments may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type and also nacreous pigments based on bismuth oxychloride. Pigments with goniochromatic properties may thus be used, especially liquid-crystal pigments or multilayer pigments. The nacres may be present in the composition in a proportion of from 0.001% to 20% and preferably in a proportion of about 1% to 15% of the total weight of the composition.

In general, the colouring agents represent from 0.001% to 60%, better still from 0.01% to 50% and even better still from 1% to 40% of the total weight of the composition.

Preferably, the colouring agent is chosen from pigments and nacres, and mixtures thereof.

According to one embodiment of the invention, the composition according to the invention also comprises at least one filler.

The term “filler” means any colourless or white particle chosen from mineral or organic, lamellar, spherical or oblong fillers, which are chemically inert in the composition. Mention may be made of talc, mica, silica, kaolin, polyamide powders, for instance Nylon® powder (Orgasol® from Atochem), poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile such as Expancel® (Nobel Industrie), acrylic polymer particles, especially of acrylic acid copolymer, for instance Polytrap® (Dow Corning) and silicone resin microbeads (for example Tospearls® from Toshiba), precipitated calcium carbonate, dicalcium phosphate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate, and mixtures thereof. These fillers may or may not be surface-treated, especially to make them lipophilic.

Preferably, the fillers have a particle size of less than 50 μm, and may represent from 0.01% to 35%, preferably from 0.05% to 25% and better still from 0.5% to 15% of the total weight of the composition, if they are present.

The composition according to the invention may advantageously comprise at least one additional non-aqueous compound other than the ester used in the composition according to the invention, chosen from oils, fatty substances that are pasty at room temperature, waxes, gums, resins and lipophilic polymers, and mixtures thereof.

This additional non-aqueous compound may represent from 0.001% to 90%, preferably from 0.05% to 60% and better still from 1% to 50% of the total weight of the composition.

In particular, it also contains at least one wax. For the purposes of the present invention, the term “wax” means a lipophilic fatty compound, which is solid at room temperature (25° C.), with a reversible solid/liquid change of state, having a melting point of greater than 30° C., which may be up to 200° C., a hardness of greater than 0.5 MPa, and having anisotropic crystal organization in the solid state. The size of the crystals is such that the crystals diffract and/or scatter light, giving the composition a more or less opaque hazy appearance. By bringing the wax to its melting point, it is possible to make it miscible with the oils and to form a microscopically uniform mixture, but, on bringing the temperature of the mixture back to room temperature, recrystallization of the wax in the oils of the mixture is obtained.

The wax may be chosen from waxes that are solid at room temperature, such as hydrocarbon-based waxes, for instance optionally modified beeswax, carnauba wax, candelilla wax, ouricoury wax, japan wax, cork fibre wax or sugarcane wax, paraffin wax, lignite wax, microcrystalline waxes, lanolin wax, montan wax, ozokerites, polyethylene wax or ethylene copolymer wax, the waxes obtained by Fischer-Tropsch synthesis, hydrogenated oils, fatty esters and glycerides that are solid at 25° C. Silicone waxes may also be used, among which mention may be made of alkyl or alkoxy polymethylsiloxanes and/or polymethylsiloxane esters. The waxes may be present in the form of stable dispersions of colloidal wax particles as may be prepared according to known methods, such as those of “Microemulsions Theory and Practice”, L.M. Prince Ed., Academic Press (1977), pages 21-32. The waxes used preferably have a melting point at least equal to 45° C.

The wax may represent from 0.01% to 50%, preferably from 2% to 40% and better still from 5% to 30% of the total weight of the composition.

The gums that may be used in the invention are generally in dissolved form in an oil and the resins may be liquid or solid at room temperature.

The nature and amount of the gums and pasty substances depend on the desired mechanical properties and textures.

The additional oils may be hydrocarbon-based oils and/or silicone oils and/or fluoro oils. These oils may be of animal, plant, mineral or synthetic origin. The term “hydrocarbon-based oil” means an oil mainly comprising carbon and hydrogen atoms, and possibly one or more functions chosen from hydroxyl, ester, ether and carboxylic functions. As examples of additional oils that may be used in the invention, mention may be made of:
hydrocarbon-based oils of animal origin such as perhydrosqualene;
hydrocarbon-based plant oils such as liquid triglycerides of fatty acids containing from 4 to 10 carbon atoms, for instance heptanoic or octanoic acid triglycerides or jojoba oil;
linear or branched hydrocarbons of mineral or synthetic origin such as liquid paraffins and derivatives thereof, and petroleum jelly;
synthetic esters and ethers, especially of fatty acids, for instance the oils of formula R₄COOR₅ in which R₄ represents an aliphatic carboxylic acid residue containing from 1 to 39 carbon atoms and R₅ represents a hydrocarbon-based chain containing from 1 to 40 carbon atoms with 10≦R₄+R₅≦41, for instance isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, and fatty alcohol heptanoates, octanoates or decanoates; certain polyol esters, for instance propylene glycol dioctanoate;
fatty alcohols containing from 12 to 26 carbon atoms, for instance octyldodecanol, 2-butyl-octanol, 2-hexyldecanol, 2-undecylpentadecanol and oleyl alcohol;
fluoro oils that are optionally partially hydrocarbon-based and/or silicone-based;
silicone oils, for instance volatile or non-volatile, linear or cyclic polydimethylsiloxanes (PDMS); polydimethylsiloxanes comprising alkyl, alkoxy or phenyl groups, which are pendant or at the end of a silicone chain, these groups containing from 2 to 24 carbon atoms; phenyl silicones, for instance phenyl trimethicones (such as the phenyl trimethicone sold under the trade name DC556 by Dow Corning), phenyl dimethicones, phenyltrimethylsiloxydiphenylsiloxanes, diphenyl dimethicones, diphenylmethyldiphenyltrisiloxanes and 2-phenyl ethyl trimethylsiloxysilicates,
and mixtures thereof.

The additional oils may represent from 0.01% to 90%, preferably from 0.05% to 60% and better still from 1% to 35% of the total weight of the composition.

The composition of the invention may also comprise at least one additive usually used in the field under consideration, such as water, antioxidants, preserving agents, neutralizers, lipophilic gelling agents or liquid non-aqueous compounds, aqueous-phase gelling agents, dispersants and cosmetic active agents, and mixtures thereof. These additives, with the exception of water, which may represent from 0 to 80%, for example from 1% to 70% and better still from 1% to 60% of the total weight of the composition, may be present in the composition in a proportion of from 0.0005% to 20% and better still from 0.001% to 10% of the total weight of the composition.

The term “cosmetic active agent” means a lipophilic or hydrophilic compound that is beneficial to keratin materials and more especially to the skin and the lips.

As cosmetic active agents that may be used in the invention, mention may be made of vitamin A, E, C, B₃ or F, provitamins, for instance D-panthenol, calmative active agents, for instance α-bisabolol, Aloe vera, allantoin, plant extracts or essential oils, protective or restructuring agents, for instance ceramides, refreshing active agents, for instance methanol and derivatives thereof, emollients (cocoa butter or dimethicone), moisturizers (arginine PCA), anti-wrinkle active agents, essential fatty acids and sunscreens, and mixtures thereof.

Needless to say, a person skilled in the art will take care to select the optional additional additives and/or the amount thereof such that the advantageous properties of the composition according to the invention are not, or are not substantially, adversely affected by the envisaged addition.

The compositions according to the invention have numerous applications and concern any coloured or uncoloured cosmetic product and more particularly lip makeup products such as lipsticks or lip glosses, and makeup products for the complexion, i.e. facial skin, such as foundations, concealers, loose or compact powders, makeup rouges, eyeshadows, blushers and makeup bases.

In one particular embodiment of the invention, the compositions according to the invention may be prepared in a manner that is usual for those skilled in the art. They may be in the form of a cast product, for example in the form of a stick or wand, or in the form of a dish that may be used via direct contact or with a sponge, or alternatively in a boiling bag. In particular, they find an application as cast foundations, cast makeup rouges or cast eyeshadows, lipsticks, lipcare sticks, bases or balms, and concealer products. They may also be in the form of a soft paste or in the form of a more or less fluid gel or cream. They may then constitute foundations, lipsticks, lip glosses, antisun products or skin colouring products.

The compositions of the invention may be anhydrous and may contain less than 5% of added water relative to the total weight of the composition. They may then especially be in the form of oily gels, oily liquids, pastes or sticks or alternatively in the form of vesicular dispersions containing ionic and/or nonionic lipids.

They may also be in the form of a simple or multiple emulsion containing an oily or aqueous continuous phase, or in the form of an oily dispersion in an aqueous phase by means of vesicles containing ionic and/or nonionic lipids. These galenical forms are prepared according to the usual methods of the fields under consideration.

The composition according to the invention may be in the form of a coloured or uncoloured skincare composition, in the form of an antisun composition or a makeup-removing composition, or alternatively in the form of a hygiene composition. If it contains cosmetic active agents, it may then be used as non-therapeutic care bases or treatment bases for the skin, for instance the hands or the face or for the lips (lip balms, for protecting the lips against cold and/or sunlight and/or wind), or an artificial skin tanning product.

The composition of the invention may also be in the form of a coloured makeup product for the skin, in particular for the face, for instance a blusher, a makeup rouge, an eyeshadow, a body makeup product, for instance a semi-permanent tattoo product or a lip makeup product, for instance a lipstick or a lip gloss, optionally having non-therapeutic care or treatment properties, a makeup product for the integuments, for instance a nail varnish, a mascara, an eyeliner, a hair colouring product or a haircare product.

Preferably, the composition according to the invention is in the form of a lip makeup product such as a lipstick or a lip gloss, or a makeup product for the complexion, i.e. for facial skin, such as a foundation.

A lip makeup product is advantageously in anhydrous form.

A complexion makeup product is preferentially in the form of an emulsion, especially an oil-in-water emulsion.

The composition according to the invention may be manufactured via the known processes generally used in cosmetics.

The examples that follow are intended to illustrate the subject of the present invention, in a non-limiting manner. The amounts are given as mass percentages.

### EXAMPLE 1

Viscosity Measurements and Spreading Properties of Compositions According to the Invention and According to the Prior Art

The inventors compared the viscosities and the spreading properties of the following compounds:
the polydimethylsiloxane manufactured or sold by the company Dow Corning under the reference Dow Corning® Fluid 200-5 cSt, which is a silicone oil commonly used in prior-art cosmetic compositions;
neopentyl glycol dineopentanoate manufactured or sold by the company BSI under the reference Neofeel 55®, which is an ester as used in the composition according to the invention.
   1.1 Viscosity Measurements

The viscosity is measured at 20° C. using a Brookfield “DV-II+” viscometer of LV type with a No. 1 spindle rotating at 100 rpm.

The results are given in Table I below:

TABLE I

| | Viscosity at 20° C. |
|---|---|
| Compound | (in cP) |
| Polydimethylsiloxane | 7.56 |
| (Dow Corning ® Fluid 200 - 5 cSt) | |
| Neopentyl glycol dineopentanoate | 11.1 |
| (Neofeel 55 ®) | |

1.2. Measurement of the Spreading Properties

A support consisting of an “ashless” filter paper of porosity 25 μm and diameter 90 mm, sold by the company Prolabo, is prepared.

0.1 ml of the test compound is then taken and placed in the centre of the filter paper, and is then left to migrate at 20° C.

After 20 minutes, the diameter of the mark obtained is measured using a millimetric ruler and the surface area of the mark is then calculated according to the following formula: surface area of the mark =(π×(diameter of the mark) 2)/4.

This surface area measurement is performed six times for the same compound. The spreading of the compound corresponds to the mean of the six surface areas obtained.

The results are given in Table (II) below.

TABLE II

| | Spreading | Standard |
|---|---|---|
| Compound | (in cm²) | deviation |
| Polydimethylsiloxane | 29.8 | 2.3 |
| (Dow Corning ® Fluid 200 - 5 cSt) | | |
| Neopentyl glycol dineopentanoate | 29 | 1.1 |
| (Neofeel 55 ®) | | |

These results show that polydimethylsiloxane 5 cSt and neopentyl glycol dineopentanoate have similar viscosities and spreading properties. It is thus possible to replace polydimethylsiloxane with neopentyl glycol dineopentanoate in a cosmetic formulation and to obtain similar cosmetic properties while at the same time having superior properties, especially in terms of migration resistance. This is demonstrated in the examples that follow.

### EXAMPLES 2 and 3

Sticks of Lipstick

The inventors compared the properties of two compositions according to the invention and the prior art as presented in Table (III). The composition of Example 2 according to the invention contains neopentyl glycol dineopentanoate and the composition of Example 3 (comparative) contains polydimethylsiloxane (5 cSt).

TABLE III

| | Example 2 | Example 3 |
|---|---|---|
| Starting materials | (invention) | (comparative) |
| Phase A | | |
| Diisostearyl malate | 4.1 | 4.1 |
| Oxypropylenated lanolin wax | 4.1 | 4.1 |
| Polybutylene | 8.2 | 8.2 |
| 2-Octyldodecanol | 4.1 | 4.1 |
| 2-Octyldodecyl neopentanoate | 0.9 | 0.9 |
| BHT | 0.04 | 0.04 |
| Phase B | | |
| Hydrogenated polyisobutene | qs 100 | qs 100 |
| (manufactured or sold under the | | |
| reference Parleam ® by the | | |
| company Nippon Oil and Fats) | | |
| Modified hectorite (Bentone ®38V | 0.8 | 0.8 |
| from Elementis) | | |
| Phase C | | |
| Polyethylene wax | 10.8 | 10.8 |
| (Mw* = 500 g/mol) | | |
| Octacosanyl stearate | 3.6 | 3.6 |
| Hydrogenated cocoglycerides | 5 | 5 |
| Phase D | | |
| Titanium dioxide | 2.7 | 2.7 |
| DC Red 7 | 2.2 | 2.2 |
| DC Red 27 aluminium lake | 1.8 | 1.8 |
| Yellow 6 aluminium lake | 3 | 3 |
| Iron oxides (CI 77499) | 2 | 2 |
| Phase E | | |
| Polydimethylsiloxane | — | 17.3 |
| (Dow Corning ® 200 Fluid - 5 cSt) | | |
| Neopentyl glycol dineopentanoate | 17.3 | — |
| (Neofeel 55 ®) | | |
| Phase F | | |
| α-Tocopheryl acetate | 0.3 | 0.3 |
| Ethylhexyl methoxycinnamate | 0.7 | 0.7 |
| Fragrance | 0.2 | 0.2 |

*Mw = molar mass

Procedure

In a first stage, the pigments (phase D) are dispersed and ground in phase A using a three-roll mill.

In parallel, a Bentone® gel (phase B) is prepared by dispersing the Bentone® in the Parleam, using a high-pressure homogenizer.

The pigments ground in phase A and the Bentone® gel are placed in a heating pan, into which are added the waxes (phase C), and the mixture is then heated at 100° C. for 2 hours and homogenized using a Rayneri mixer.

Phase E and the active agents (phase F) are then added and the mixture is homogenized for 5 minutes and then poured into a suitable mould at 42° C. to form sticks.

The mould is then placed at −20° C. for half an hour and the sticks are then removed from the mould.

Sensory Evaluation

The compositions of Examples 2 and 3 were applied, by application to half a lip, to a panel of 5 qualified individuals and evaluated according to various criteria.

The grades for the various criteria are between 0 (none) and 10 (substantial) inclusive.

The results are given in Table (IV) below.

TABLE IV

Results

| | Example 2 | Example 3 |
|---|---|---|
| Evaluated criteria | (Invention) | (Comparative) |
| On applying the stick to the lips | | |
| slipperiness | 6.4 | 7 |
| oily/greasy | 4.8 | 4.8 |
| softness | 7.8 | 7.4 |
| dry | 2.8 | 2.6 |
| tack | 1.8 | 2 |
| thickness of the film | 4.5 | 4.8 |
| uniformity of the film | 7 | 7 |
| gloss | 6.8 | 5.6 |
| coverage** | 6.6 | 7 |
| After application | | |
| gloss | 5.4 | 5.4 |
| tack | 2.2 | 2 |
| pleasantness of application | 7.1 | 7 |
| makeup result | 6.8 | 6.6 |
| quality of the product | 7.1 | 6.9 |
| After one hour | | |
| comfort | 5.8 | 6.2 |
| tack | 2.4 | 2.1 |
| staying power of the film | 6.8 | 6.5 |
| migration | 2.8 | 3.8 |
| gloss | 3.4 | 3.2 |

(**the coverage is the ability of a makeup composition to cover the support onto which the said composition is applied, especially to hide the imperfections thereof).

The composition of Example 2 has better migration-resistance properties than those of the composition of Example 3 (comparative), the other cosmetic criteria being equivalent to those of the composition containing polydimethylsiloxane

### COMPARATIVE EXAMPLE 3.

The composition of Example 2 especially has excellent properties in terms of slipperiness, softness, coverage and pleasantness of application, and also comfort and staying power of the film of composition over time.

It also has good gloss properties on application and after application.

Neopentyl glycol dineopentanoate can thus advantageously replace polydimethylsiloxane in cosmetic compositions to obtain the properties mentioned above.

### EXAMPLE 4

Stick of Lipstick

A composition according to the invention containing glyceryl trineopentanoate manufactured or sold by the company BSI under the reference Neofeel TNP® is prepared according to the procedure of Examples 2 and 3.

| | |
|---|---|
| Diisostearyl malate | 4.1 |
| Oxypropylenated lanolin wax | 4.1 |
| Polybutylene | 8.2 |
| 2-Octyldodecanol | 4.1 |
| 2-Octyldodecyl neopentanoate | 0.9 |
| BHT | 0.04 |
| Hydrogenated polyisobutene (manufactured or | qs 100 |
| sold under the reference Parleam ® by the | |
| company Nippon Oil and Fats) | |
| Modified hectorite (Bentone ® 38V from | 0.8 |
| Elementis) | |
| Polyethylene wax (Mw* = 500 g/mol) | 10.8 |
| Octacosanyl stearate | 3.6 |
| Hydrogenated cocoglycerides | 5 |
| Titanium dioxide | 2.7 |
| DC Red 7 | 2.2 |
| DC Red 27 lake | 1.8 |
| Yellow 6 lake | 3 |
| Iron oxides (CI 77499) | 2 |
| Glyceryl trineopentanoate (manufactured or sold | 17.3 |
| by the company BSI under the reference Neofeel | |
| TNP ®) | |
| α-Tocopheryl acetate | 0.3 |
| Ethylhexyl methoxycinnamate | 0.7 |
| Fragrance | 0.2 |

*Mw = molar mass

A stick of lipstick that allows the deposition of a comfortable, non-irritant film of lipstick with good staying power, which migrates little or not at all, is obtained.

### EXAMPLE 5

Water-in-Oil foundation

A foundation composition containing, as ester according to the invention, the glyceryl trineopentanoate manufactured or sold by the company BSI under the reference Neofeel TNP® is prepared.

| Oily phase | |
|---|---|
| Glyceryl trineopentanoate (manufactured or sold | 29.4 |
| by the company BSI under the reference | |
| Neofeel TNP ®) | |
| Cetyldimethicone copolyol (manufactured or sold | 3 |
| by the company Goldschmidt under the reference | |
| Abil ® EM 90) | |
| Mixture of acetylated glycol stearate and of | 0.5 |
| tristearine (manufactured or sold by the | |
| company Guardian under the reference Unitwix ®) | |
| Propyl paraben | 0.1 |
| Aqueous phase | |
| Water | 57.2 |
| Magnesium sulfate | 0.7 |
| Glycerol | 5 |
| Methyl paraben | 0.1 |
| Pigments | |
| Yellow iron oxide coated with isopropyl | 0.6 |
| triisostearyl titanate (ITT) | |
| ITT-coated red iron oxide | 0.39 |
| ITT-coated black iron oxide | 0.11 |
| ITT-coated titanium dioxide | 2.9 |

Procedure

The pigments are dispersed in 4% of glyceryl trineopentanoate using a three-roll mill (the pigments are milled 3 times).

The rest of the constituents of the oily phase are weighed out together and then heated to 70° C.

The ground and then homogenized pigments are added to the oily phase with stirring using a Moritz stirrer (at a speed of 1000 rpm).

In parallel, the constituents of the aqueous phase are weighed out together and then heated to 80° C.

The aqueous phase is slowly added to the mixture of oily phase +pigments while maintaining a minimum temperature of 50° C. during the addition, and changing to a stirring speed of 3000 rpm.

The mixture is then allowed to cool gradually to room temperature with continued stirring.

The foundation obtained is in the form of a cream. It has good staying power properties while at the same time being comfortable.

1. Composition comprising a physiologically acceptable medium containing a fatty phase comprising at least one ester resulting from the reaction of a polyol with a carboxylic acid of formula (I) below: 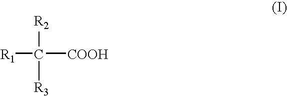
in which R₁, R₂ and R₃ are radicals independently chosen from optionally functionalized alkyl, aryl and aralkyl radicals, and combinations thereof,
characterized in that the said polyol is chosen from polyols comprising one carbon atom, located alpha to the carbon bearing an alcohol function, which is trisubstituted with radicals chosen, independently of each other, from alkyl, aryl and aralkyl radicals and combinations thereof, at least one of the alkyl, aryl and aralkyl radicals containing at least one alcohol function, the said polyol not being 2,2,4-trimethyl-1,3-pentanediol.

2. Composition comprising a physiologically acceptable medium containing a fatty phase comprising at least one ester resulting from the reaction of a polyol with a carboxylic acid of formula (I) below: 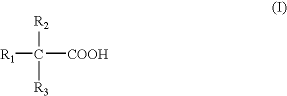
in which R₁, R₂ and R₃ are radicals independently chosen from optionally functionalized alkyl aryl and aralkyl radicals, and combinations thereof,
the said acid containing from 5 to 9 carbon atoms, and the said polyol not being a compound of formula HO(CₙH₂ₙO)ₘH such that n is equal to 2 or 3 and m is between 2 and 4.

3. Composition comprising a physiologically acceptable medium containing a fatty phase comprising at least one ester resulting from the reaction of a polyol with a carboxylic acid of formula (I) below: 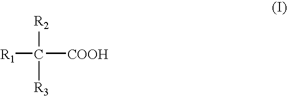
in which R₁, R₂ and R₃ are radicals independently chosen from optionally functionalized alkyl, aryl and aralkyl radicals, and combinations thereof,
the said ester comprising at least three ester functions.

4. Composition according to one of the preceding claims, characterized in that the radicals R₁, R₂ and R₃ are chosen, independently of each other, from saturated alkyl radicals.

5. Composition according to one of the preceding claims, characterized in that the carboxylic acid is a monoacid.

6. Composition according to one of the preceding claims, characterized in that the polyol is a diol, a triol or a tetraol.

7. Composition according to claim 1 or 3, characterized in that the radicals R₁, R₂ and R₃ are chosen, independently of each other, from saturated C₁-Cl₅ alkyl radicals.

8. Composition according to claim 7, characterized in that the radicals R₁, R₂ and R₃ are chosen, independently of each other, from saturated C₁-C₆ alkyl radicals.

9. Composition according to claim 1 or 3, characterized in that the carboxylic acid comprises a total number of carbon atoms ranging from 5 to 30, preferably from 5 to 15 and preferably from 5 to 10.

10. Composition according to claim 1 or 3, characterized in that the carboxylic acid is chosen from neopentanoic acid, neohexanoic acid, neoheptanoic acid and neodecanoic acid, and mixtures thereof.

11. Composition according to claim 2, characterized in that the carboxylic acid contains from 5 to 7 carbon atoms.

12. Composition according to claim 11, characterized in that the carboxylic acid is chosen from neopentanoic acid, nebhexanoic acid and neoheptanoic acid, and mixtures thereof.

13. Composition according to claim 1, characterized in that the polyol comprises a number of carbon atoms ranging from 5 to 20 and preferably from 5 to 10.

14. Composition according to claim 1, characterized in that the polyol contains a carbon atom located alpha to the carbon bearing one of the alcohol functions that is trisubstituted with radicals independently chosen from saturated alkyl radicals, at least one of the alkyl radicals containing at least one alcohol function.

15. Composition according to the preceding claim, characterized in that the saturated alkyl radicals are of C₁-C₁₅.

16. Composition according to the preceding claim, characterized in that the saturated alkyl radicals are of C₁-C₆.

17. Composition according to claim 16, characterized in that the polyol is chosen from trimethylolpropane and pentaerythritol, and mixtures thereof.

18. Composition according to claim 16, characterized in that the polyol is neopentyl glycol.

19. Composition according to claim 2 or 3, characterized in that the polyol comprises a number of carbon atoms ranging from 2 to 20 and preferably from 3 to 10.

20. Composition according to claim 2, characterized in that the polyol is chosen from ethylene glycol, propylene glycol, butylene glycol, polyethylene glycols other than a compound of formula HO(CₙH₂ₙO)ₘH such that n is equal to 2 or 3 and m is between 2 and 4, polypropylene glycols, glycerol, diglycerol, triglycerol, isopentyldiol and sorbitol, and mixtures thereof.

21. Composition according to claim 3, characterized in that the polyol is chosen from ethylene glycol, propylene glycol, butylene glycol, polyethylene glycols, polypropylene glycols, glycerol, diglycerol, triglycerol, isopentyldiol and sorbitol, and mixtures thereof.

22. Composition according to claim 1, characterized in that the ester is chosen from neopentyl glycol dineopentanoate and neopentyl glycol dineoheptanoate.

23. Composition according to claim 2 or 3, characterized in that the ester is chosen from glyceryl trineopentanoate and glyceryl trineoheptanoate.

24. Composition according to claim 3, characterized in that the ester is glyceryl trineodecanoate.

25. Composition according to one of the preceding claims, characterized in that the said ester is in liquid form at room temperature (25° C.).

26. Composition according to one of the preceding claims, characterized in that the ester represents from 0.1% to 99.9%, preferably from 1% to 99% and better still from 5% to 90% of the total weight of the composition.

27. Composition according to one of the preceding claims, characterized in that the ester is in an amount that is sufficient to give the composition gloss and/or staying power and/or migration resistance and/or comfort properties.

28. Composition according to one of the preceding claims, characterized in that it also comprises at least one colouring agent.

29. Composition according to one of the preceding claims, characterized in that the colouring agent represents from 0.001% to 60%, better still from 0.01% to 50% and even better still from 0.1% to 40% of the total weight of the composition.

30. Composition according to one of the preceding claims, characterized in that it also comprises at least one filler.

31. Composition according to one of the preceding claims, characterized in that the filler represents from 0.01% to 35%, preferably from 0.05% to 25% and better still from 0.5% to 15% of the total weight of the composition.

32. Composition according to one of the preceding claims, characterized in that it comprises at least one additional non-aqueous compound chosen from oils, fatty substances that are pasty at room temperature, waxes, gums, resins and lipophilic polymers, and mixtures thereof.

33. Composition according to one of the preceding claims, characterized in that the additional non-aqueous compounds represent from 0.001% to 90%, preferably from 0.05% to 60% and better still from 1% to 50% of the total weight of the composition.

34. Composition according to one of the preceding claims, characterized in that it also comprises at least one wax.

35. Composition according to one of the preceding claims, characterized in that the wax represents from 0.01% to 50%, preferably from 2% to 40% and better still from 5% to 30% of the total weight of the composition.

36. Composition according to one of the preceding claims, characterized in that it is in the form of a makeup and/or care product for the face or the body, the lips and/or the integuments.

37. Composition according to one of the preceding claims, characterized in that it is in the form of a makeup product for facial skin.

38. Composition according to one of the preceding claims, characterized in that it is in the form of a lip makeup product.

39. Composition according to the preceding claim, characterized in that it is in anhydrous form.

40. Composition according to the preceding claim, characterized in that it is in the form of an emulsion, such as an oil-in-water or water-in-oil emulsion.

41. Use of at least one ester resulting from the reaction of a polyol with a carboxylic acid of formula (I) below: 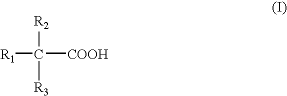
in which R₁, R₂ and R₃ are radicals independently chosen from optionally functionalized alkyl, aryl and aralkyl radicals, and combinations thereof,
characterized in that the said polyol is chosen from polyols comprising one carbon atom, located alpha to the carbon bearing an alcohol function, which is trisubstituted with radicals chosen, independently of each other, from alkyl, aryl and aralkyl radicals and combinations thereof, at least one of the alkyl, aryl and aralkyl radicals containing at least one alcohol function, the said polyol not being 2,2,4-trimethyl-1,3-pentanediol,
in a cosmetic composition as an agent for giving the said composition staying power properties, especially of the colour, and/or gloss and/or comfort and/or migration resistance properties.

42. Use of at least one ester resulting from the reaction of a polyol with a carboxylic acid of formula (I) below: 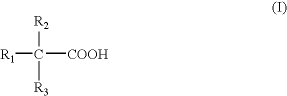
in which R₁, R₂ and R₃ are radicals independently chosen from optionally functionalized alkyl, aryl and aralkyl radicals, and combinations thereof,
characterized in that the said polyol is chosen from polyols comprising one carbon atom, located alpha to the carbon bearing an alcohol function, which is trisubstituted with radicals chosen, independently of each other, from alkyl, aryl and aralkyl radicals, and combinations thereof, at least one of the alkyl, aryl and aralkyl radicals containing at least one alcohol function, the said polyol not being 2,2,4-trimethyl-1,3-pentanediol,
in a cosmetic composition with staying power properties, especially of the colour, and/or gloss and/or comfort and/or migration resistance properties.

43. Use of at least one ester resulting from the reaction of a polyol with a carboxylic acid of formula (I) below: 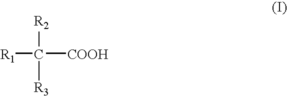
in which R₁, R₂ and R₃ are radicals independently chosen from optionally functionalized alkyl aryl and aralkyl radicals, and combinations thereof,
the said acid containing from 5 to 9 carbon atoms, and the said polyol not being a compound of formula HO(CₙH₂ₙO)ₘH such that n is equal to 2 or 3 and m is between 2 and 4,
in a cosmetic composition as an agent for giving the said composition staying power properties, especially of the colour, and/or gloss and/or comfort and/or migration resistance properties.

44. Use of at least one ester resulting from the reaction of a polyol with a carboxylic acid of formula (I) below: 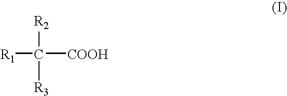
in which R₁, R₂ and R₃ are radicals independently chosen from optionally functionalized alkyl aryl and aralkyl radicals, and combinations thereof, the said acid containing from 5 to 9 carbon atoms, and the said polyol not being a compound of formula HO(CₙH₂ₙO)ₘH such that n is equal to 2 or 3 and m is between 2 and 4
in a cosmetic composition with staying power properties, especially of the colour, and/or gloss and/or comfort and/or migration resistance properties.

45. Use of at least one ester resulting from the reaction of a polyol with a carboxylic acid of formula (I) below: 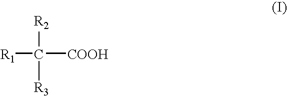
in which R₁, R₂ and R₃ are radicals independently chosen from optionally functionalized alkyl aryl and aralkyl radicals, and combinations thereof,
the said ester comprising at least three ester functions,
in a cosmetic composition as an agent for giving the said composition staying power properties, especially of the colour, and/or gloss and/or comfort and/or migration resistance properties.

46. Use of at least one ester resulting from the reaction of a polyol with a carboxylic acid of formula (I) below: 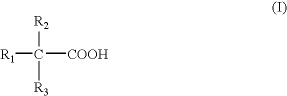
in which R₁, R₂ and R₃ are radicals independently chosen from optionally functionalized alkyl, aryl and aralkyl radicals, and combinations thereof,
the said ester comprising at least three ester functions,
in a cosmetic composition with staying power properties, especially of the colour, and/or gloss and/or comfort and/or migration resistance properties.
